(19) United States
(12) Patent Application Publication
(10) Pub. No.: US 2009/0131499 A1
(43) Pub. Date: May 21, 2009

(54) PHOTODYNAMIC THERAPY FOR SKIN RELATED PROBLEMS

(75) Inventors: Danilo Castro, Montevideo (UY); Wolfgang Neuberger, F.T. Labuan (MY)

Correspondence Address: Dr. Bolesh J. Skutnik;CeramOptec Industries, Inc., 515 Shaker Road, East Longmeadow, MA, 01028, US

(73) Assignee: CERAMOPTEC INDUSTRIES INC.

(21) Appl. No.: 12/271,708

(22) Filed: Nov. 14, 2008

Related U.S. Application Data
(60) Provisional application No. 61/003,563, filed on Nov. 15, 2007.

(51) Int. Cl. A61K 31/407

(52) U.S. Cl. 514/410

(57) ABSTRACT

PhotoDynamic Therapy method is used to treat problems associated with pilosebaceous units including acne and other skin conditions. At least one liposomal-formulated, hydrophobic photosensitizer, such as a dihydro- or tetrahydro-porphyrin, is topically applied to acne affected regions, and is allowed to penetrate the hair follicle before being irradiated with a suitable wavelength of light energy overlapping the absorption spectrum of the selected photosensitizer. The photoactivated agent initiates a cytotoxic effect in the sebaceous gland, reducing excess oil production and destroying bacterial growth in the follicle thus subsiding inflammatory action. Scarring of the skin is minimized and the skin heals faster due to collagen cell stimulation.

### DOMESTIC PRIORITY UNDER 35 USC 119(e)

This application claims the benefit of U.S. Provisional Application Ser. No. 61/003,563 filed Nov. 15, 2007, entitled “PhotoDynamic Therapy for Skin Related Problems” by Wolfgang Neuberger and Danilo Castro, which is incorporated by reference herein.

### BACKGROUND OF THE INVENTION

1. Field of the Invention

The present invention relates to photodynamic therapy (PDT) in general, and in particular, to use of PDT methods for treating moderate to severe acne conditions in skin and skin-related problems.

2. Invention Disclosure Statement

The skin is a major organ performing various functions for the body and is exposed to highly extreme conditions. Among the major dermatological problems, acne is the most common, affecting nearly 80 percent of all American teenagers and adults. Acne is a disorder of the pilosebaceous units. A pilosebaceous unit is made up of a hair follicle, sebaceous gland, and hair shaft. Sebaceous glands produce sebum, which lubricates the skin and provides a protective barrier to prevent drying of upper skin layers. During adolescence, sebaceous glands enlarge and produce more sebum under the influence of hormones called androgens.

The three major factors responsible for acne development are: overproduction of oil by enlarged oil glands in the skin; blockage of the hair follicles and growth of Propionibacterium acnes. P. acnes is a normal inhabitant of the skin. Increased populations of P. acnes are common in acne lesions. P. acnes grows in the hair follicle and is nourished by sebum. The toxic byproducts of P. acnes in the gland attract immune cells to the follicle. Due to this inflammatory response, papules, pustules and nodules are commonly seen on the skin.

Acne lesions can be of a non-inflammatory or inflammatory type. Non-inflammatory acne lesions include blackheads (open comedones) and whiteheads (closed comedones). Open or closed comedones, along with papules and pustules are referred to as papulopustular acne—a form of inflammatory acne. Nodular acne is the most severe form of inflammatory acne. Lesions are found in areas where there are greater concentrations of sebaceous glands, e.g., the face, neck and upper part of the trunk.

A variety of topical and systemic medications are currently available for acne treatment. Topical medications can be in the form of a cream, a gel, a lotion or a solution of one or more of benzoyl peroxide, antibiotics, tretinion and adapalene. Systemic or oral antibiotics are often prescribed to treat moderate to severe acne. The effectiveness of antibiotics is reduced due to rise in bacterial resistance. Isotretinoin is a very effective agent used to treat severe, cystic and inflammatory acne conditions. Isotretinoin is reported to cause severe birth defects and hence its use needs to be carefully monitored by a physician. As a result, research continues for finding efficient acne treatment agents with minimal side effects.

Several new acne treatment techniques using new lasers and light energy have been proposed. Different types of light energy are used to treat acne, for example ultraviolet (UV) light, with wavelengths of 320 nanometers (nm) and 350 nm, is used in acne treatment. UV light is unsuitable as a long-term remedy, however, because of increased risks of skin cancer, and also has adverse effects like aging (by UV-A) and burning (by UV-B).

In U.S. Patent Application No. 2008/0172045A1, Shanks et. al describe a device that contains light emitting devices capable of impinging a treatment area with laser light. In the preferred embodiment, the light emitting devices emit a combination of red and blue light to treat acne causing bacteria destruction and prevent inflammation.

In U.S. Pat. No. 6,887,260 by McDaniel, a method and apparatus are disclosed for treating skin disorders, such as acne, by administering the sebaceous gland and surrounding tissue with a topical chromophore composition selected from the group consisting of chlorophyll, porphyrin, carotenoids, bacteriophyll, phycobilins, and its combinations having absorption maximum between 300 nm and 1300 nm (ultraviolet, visible or infrared region of the spectrum) and a total energy fluence less than four Joules per square centimeter (J/cm²) to stop the excess in oil production and kill bacterial growth in the region. The primary use of wavelengths in UV region can increase the risk of skin cancer and also cause photo-damage to the skin cells.

A narrow band of FDA-approved blue light has proven highly effective in the treatment of acne. In this method the endogenously produced porphyrins are excited by bluelight to produce reactive oxygen species (ROS) that destroy the bacterial growth and reduce oil production to prevent acne formation. U.S. Pat. No. 6,835,202, by Korman et al., discloses a method and apparatus used in PDT treatment for skin disorders like acne vulgaris and seborrhea. In this invention, spectral emission in the range between 400 nm and 450 nm is used to photoactivate photosensitive agents such as methylene blue solution to bring about a cytotoxic effect in the target cells.

In U.S. Pat. No. 6,897,238, Anderson describes a PDT for treating acne by topical application of aminolevulinic acid (ALA), followed by light irradiation. The low dose ALA-PDT shows reduction in acne formation by reducing oil production and bacterial growth, while high dose ALA-PDT can cause permanent changes to the sebaceous gland. Use of high light dosage can cause damage to skin; leading to scaring, and hence would require a cooling system. Permanent changes in the sebaceous gland can cause dry skin problems if treated too harshly.

Use of PDT methods in acne reduction may also cause a sunburn-like reaction, such as redness and light peeling, for a few days following the treatment. Depending on the treatment aggressiveness, other effects like swelling and hyper-pigmentation are common. Most of the prior art treatments might require multiple sittings, long term post-treatment skin care and regular maintenance. Similarly, use of high fluence light energy can cause damage to the eyes, hence safety precautions need to be taken.

The present invention overcomes most of the side effects cited in the above treatment methods. It provides faster, safer treatment methods for mild to severe acne conditions with minimal side effects and shorter healing time.

### OBJECTIVES AND BRIEF SUMMARY OF THE INVENTION

It is an objective of the present invention to effectively treat pilosebaceous gland disorders, including acne and other skin problems.

It is another objective of the present invention to use Photodynamic Therapy (PDT) for treating severe to moderate acne conditions without the harmful effect of UV rays.

It is still another objective of the present invention to reduce oil production, bacterial growth and to expedite the healing process.

It is also the objective of the present invention to limit or eradicate moderate to severe acne conditions, to prevent scaring and to improve skin appearance.

Briefly stated, in the present invention a PhotoDynamic Therapy method is presented for use to treat problems associated with pilosebaceous units including acne and other skin conditions. At least one liposomal-formulated, hydrophobic photosensitizer, such as a dihydro- or tetrahydro-porphyrin, is topically applied to acne affected regions, and is allowed to penetrate the hair follicle before being irradiated with a suitable wavelength of light energy overlapping the absorption spectrum of the selected photosensitizer. The photoactivated agent initiates a cytotoxic effect in the sebaceous gland, reducing excess oil production and destroying bacterial growth in the follicle thus subsiding inflammatory action. Scarring of the skin is minimized and the skin heals faster due to collagen cell stimulation.

The above and other objects, features and advantages of the present invention will become apparent from the following description read in conjunction with the accompanying drawing(s).

### BRIEF DESCRIPTION OF FIGURES

FIG. 1 is a diagram illustrating an exemplary methodology for PDT treatment of acne.

### DETAILED DESCRIPTION OF PREFERRED EMBODIMENTS

Acne is an inflammatory disease of the skin, caused by changes in the pilosebaceous units. Acne lesions are commonly referred to as pimples, spots or zits. More than 70-80 percent of Americans are affected by acne. It is most common in puberty. Acne can cause emotional and physical concern to these young patients when it cannot be treated.

Three major factors that cause acne are the overproduction of oil by enlarged oil glands in the skin; blockage of the hair follicles that release the oil; and a growth of bacteria within the hair follicles. At present, different methods are used for treating and controlling acne which includes oral antibiotic, topical application, lasers and the latest being Photodynamic Therapy (PDT). Depending on the skin type and acne condition, different types of treatment are employed. Skin types are determined based on Fitzpatrick classification which is determined by genetics and reaction of the skin to sun exposure and tanning habits. PDT is a newer treatment modality used in treating certain types of cancer, wherein, the hyperproliferative tissues are pre-sensitized with photosensitive agents followed by irradiation with light to induce tissue photodestruction.

The present invention uses PDT to treat inflammatory and non-inflammatory types of acne without exposure to ultraviolet (UV) light. In general, the method involves administering a photosensitizer(s) suitably formulated for better drug action and better accumulation in the affected sebaceous gland. After a predetermined time interval, light energy is radiated at this region in order to induce photodestruction. This method reduces overproduction of oil by enlarged glands, kills bacteria and reduces the pore size.

Suitable photosensitizers include photoactive agents and their derivatives suitably formulated into a drug delivery system which can include, gels, liposomes, nanoparticles, pegylation, microspheres or other polymer carriers.

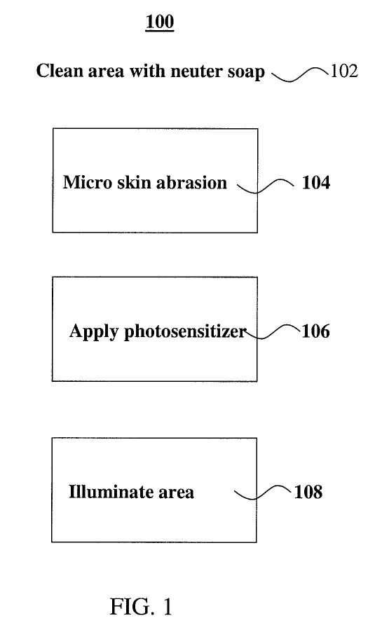

FIG. 1 is a diagram illustrating exemplary methodology 100 for PDT treatment of acne. In step 102, the skin around the affected area is cleaned, for example, using a mild cleansing agent, such as a pH neutral soap.

In step 104, a micro skin abrasion procedure is performed, e.g., using a Diamond peel, to open the skin pores especially in the region of acne spots within the affected area. The opening of the pores facilitates transport of the topically-applied photosensitizer (see step 106, described below).

In step 106, a photosensitizer is topically applied to the treatment areas. According to an exemplary embodiment, the photosensitizer comprises gel formulated temoporfin (mTHPC), and is applied individually to each acne spot.

In step 108, about 20 minutes after drug application, the treated areas are illuminated for a duration of up to about 30 minutes with a special lamp Hydrosun irradiator PhotoDyn 505 with infrared barrier filter.

This procedure is performed once a week for up to about three to four weeks, as needed, and it depends, in part, on the patient's recovery and improvement in skin condition.

In a preferred embodiment, temoporfin (tetra [m-hydroxyphenyl] chlorin (m-THPC)) enclosed in liposomal vesicle is used for treating inflammatory and non-inflammatory acne conditions. The gel formulation of temoporfin is locally administered into the affected skin regions, followed by a short, predetermined time interval (drug-light interval, DLI) for accumulation of the drug in the affected pilosebaceous unit with bacterial infection. This is followed by irradiation of the acne with laser energy, at wavelengths in visible region, to activate the temoporfin within the affected cells and near bacteria so as to induce photodestruction without harming the surrounding regions in the dermal layer of skin, thus decreasing pore size and reducing oil production to prevent further clogging of the pore and bacterial growth. The treated regions heal faster with minimal scaring and better skin tone, than observed in prior treatment modalities. Partial destruction of oil gland helps to reduce the acne considerably without causing dryness.

The present invention is further illustrated by the following examples, but is not limited thereby.

### EXAMPLE 1

### For Treating Inflammatory Type of Acne

Severe inflammatory or cystic acne which has failed all conventional treatment can be treated by PDT method in the following manner. Before applying photosensitizer, the entire face of the patient is washed clean. Gel formulated temoporfin is locally applied to the acne lesion and after a predetermined time interval of generally about 20 minutes, the targeted region is illuminated for 30 minutes with a special lamp Hydrosun irradiator PhotoDyn 505 with infrared barrier filter. Continuous light is used for illuminating the acne spots. The light fluence used is 10 J/cm².

The photodynamic action destroys the microbial growth in the sebaceous gland, reducing the acne lesion and its associated inflammation. The hyperactive old gland is partially destructed, thus reducing the excess in oil production. The open pores are partially closed, thus preventing bacterial growth and further acne production. Patients examined after a week showed a progressive reduction in the number of lesions with minimal scaring and improved skin texture.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A PhotoDynamic therapy (PDT) method for treating moderate to severe acne conditions and other related skin problems, the method comprising the steps of:
cleansing skin in an acne-affected area;
performing a micro-skin abrasion to open skin pores in said affected area;
topically applying a photosensitizer formulation to said affected area;
waiting for a predetermined time to allow formulation to penetrate pores in said affected area; and
irradiating said affected area, with a wavelength compatible with said photosensitizer formulation.

2. The PDT method according to claim 1, wherein the step of cleansing said acne skin is performed using a pH neutral soap.

3. The PDT method according to claim 1, wherein the said micro-skin abrasion is performed with diamond peel.

4. The PDT method according to claim 3, wherein the said micro skin abrasion opens skin pores near acne spots.

5. The PDT method according to claim 1, wherein photochemical formulation contains at least one hydrophobic photosensitizer from the group of di-hydro or tetra-hydro-porphyrins.

6. The PDT method according to claim 5, wherein said at least one hydrophobic sensitizer is temoporfin (m-THPC).

7. The PDT method according to claim 5, wherein said photosensitizer formulation is a gel formulation.

8. The PDT method according to claim 5, wherein the said irradiating step is performed about 20 minutes after gel application.

9. The PDT method according to claim 1, wherein the said irradiating step is performed on the skin for a duration of about 30 minutes.

10. The PDT method according to claim 9, wherein the said irradiating step is performed using continuous light.
